(19) United States
(12) Patent Application Publication
(10) Pub. No.: US 2023/0395194 A1
(43) Pub. Date: Dec. 7, 2023

(54) DETECTION OF DELETIONS IN OLIGONUCLEOTIDE SEQUENCES

(71) Applicant: GENIEUS GENOMICS PTY LTD, Darlinghurst (AU)

(72) Inventors: Ted Wong, Darlinghurst (AU); Zheng Su, Darlinghurst (AU); Matthew Keon, Darlinghurst (AU); Boris Guennewig, Darlinghurst (AU)

(21) Appl. No.: 18/250,117

(22) PCT Filed: Oct. 20, 2021
(86) PCT No.: PCT/AU2021/051220

§ 371 Date: Apr. 21, 2023

(30) Foreign Application Priority Data
Oct. 23, 2020 (AU) AU2020903839

(51) Int. Cl. G16B 30/00; G16B 40/00

(52) U.S. Cl. CPC G16B 30/00; G16B 40/00

(57) ABSTRACT

Disclosed herein is a method for detecting deletion in a gene sequence. The method comprises receiving, by a processor, training sequencing data, which comprises multiple training reads associated with gene sequences with deletion and gene sequences without deletion. The processor splits each of the multiple training reads into multiple training segments shorter than the training reads and trains a machine learning model on the multiple segments. The processor receives testing sequencing data comprising multiple testing reads, splits each of the multiple testing reads into multiple testing segments, and evaluates the trained machine learning model to the multiple testing segments to detect deletion in the testing sequencing data. No alignment or variant calling is necessary, which reduces the computational complexity of the evaluation step significantly.

### CROSS-REFERENCE TO RELATED APPLICATIONS

The present application claims priority from Australian provisional application 2020903839, the contents of which are incorporated herein by reference in their entirety.

### TECHNICAL FIELD

This disclosure relates to detecting deletions in a genome.

### BACKGROUND

The analysis of the entire human genome has been facilitated in recent years by the introduction of sequencing by synthesis, where a large number of relatively short fragments of DNA, RNA or other oligonucleotide sequences are read in parallel. These ‘reads’ are then often aligned against a reference genome in order to detect variants, such as single nucleotide polymorphisms where one nucleotide base is changed to a different base.

Another form of variants are structural variants, which include deletions. However, the detection of deletions from short reads is difficult since the deleted region is often longer than a single read, which makes the alignment process computationally expensive and inaccurate.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY

This disclosure provides a method for detecting deletions where, instead of aligning the short reads, each read is split into segments of length k, also referred to as k-mers or simply mers. The proposed method then trains a machine learning model directly on the k-mers without alignment. In case of a deletion, the method can then detect the absence of the deleted k-mers and the presence of k-mers that are missing the parts that belong to the deleted DNA sequence. As a result, diseases that are associated with such deletions, can be diagnose accurately.

Disclosed herein is a computer-implemented method for detecting deletion in a gene sequence. The method comprises receiving training sequencing data, the training sequencing data comprising multiple training reads associated with gene sequences with deletion and gene sequences without deletion, splitting each of the multiple training reads into multiple training segments shorter than the training reads, training a machine learning model on the multiple segments, receiving testing sequencing data comprising multiple testing reads, splitting each of the multiple testing reads into multiple testing segments, and evaluating the trained machine learning model to the multiple testing segments to detect deletion in the testing sequencing data.

It is an advantage that the method trains and evaluates the machine learning model on multiple segments of the sequence. As a result, no alignment or variant calling is necessary, which reduces the computational complexity of the evaluation step significantly. It is noted that the training step may be computationally expensive, but this step is only performed once for the entire training data set.

In some embodiments, the training segments and the testing segments are k-mers.

In some embodiments, the testing sequencing data is generated by a sequencer. In some embodiments, the testing sequencing data is provided in a FASTQ file from the sequencer.

In some embodiments, the machine learning model is a neural network. In some embodiments, the neural network comprises a gated recurrent unit. In some embodiments, the neural network comprises a bidirectional gated recurrent unit to process forward and reverse read directions of the training sequencing data and the testing sequencing data. In some embodiments, the method further comprises encoding the segments and using the encoded segments directly as an input to the bidirectional gated recurrent unit.

In some embodiments, the method further comprises performing one or more steps of the method on a graphics processing unit.

In some embodiments, the method further comprises detecting a disease based on the deletion.

In some embodiments, detecting the disease is an output of the trained machine learning model.

In some embodiments, the training sequencing data and the testing sequencing data is obtained by sequencing by synthesis.

In some embodiments, the training sequencing data and the testing sequencing data comprise RNA reads and the deletion is in a genome of a subject.

In some embodiments, the reads are between 100 and 200 base pairs long and the segments are between 4 and 100 base pairs long.

In some embodiments, the segments are between 4 and 20 base pairs long.

Software, when executed by a computer, causes the computer to perform the method above.

There is further disclosed a computer system for detecting deletion in a gene sequence. The computer system comprises data memory configured to store training sequencing data, the training sequencing data comprising multiple training reads associated with gene sequences with deletion and gene sequences without deletion, a processor configured to split each of the multiple training reads into multiple training segments shorter than the training reads, train a machine learning model on the multiple segments, receive testing sequencing data comprising multiple testing reads, split each of the multiple testing reads into multiple testing segments, and evaluate the trained machine learning model to the multiple testing segments to detect deletion in the testing sequencing data.

### BRIEF DESCRIPTION OF DRAWINGS

An example will now be described with reference to the following drawings:

FIG. 1 illustrates a computer system for detecting deletions in a genome.

FIG. 2 illustrates a method for detecting deletion in a DNA sequence.

FIG. 3 illustrates a machine learning model including an embedding layer.

FIG. 4 illustrates a machine learning model with direct input into the gate recurrent unit.

FIG. 5 illustrates a sigmoid curve.

### DESCRIPTION OF EMBODIMENTS

### System

FIG. 1 illustrates a computer system 100 for detecting deletions in a genome. Computer system 100 comprises a processor 101, program memory 102, data memory 103, a communication port 104, a graphics processing unit (GPU) 105 and a database 106. System 100 is connected through communication port 104 to a sequencer 110, which comprises a flow cell 111 onto which multiple strands of oligonucleotides 112 are connected and a camera 113 to capture fluorescent labels attached to the strands 112. In one example, the sequencer 110 performs sequencing by synthesis, whereby in each cycle one label is attached to each strand 112 depending on which base is at the current position in the strand 112. The labels for each base are fluorescent at different colours so that the camera 113 captures an image where each coloured dot in the image represents one of the bases. Processor 101 can then perform a base calling method to determine the base for each cycle and concatenate the bases from each strand into a ‘read’. In one example, sequencer 110 is an X10 next generation sequencing (NGS) sequencer by Illumina Inc.

It is noted that processor 101 may receive the image data from sequencer 110 or may receive the base calls from sequencer 110. In the latter case, sequencer 110 performs the base calling internally and provides a FASTQ file containing the bases and further quality information, for example. Any data received from sequencer 110 that is indicative of bases or nucleotides is referred to as sequencing data. Processor 101 uses the sequencing data to detect deletions in a gene sequence.

A deletion is a type of variant of DNA. Other types include single nucleotide polymorphisms (SNPs), where a single base is changed. SNPs can be detected by aligning the reads to a reference genome and determining the difference between the reads and the reference genome. For deletions, however, alignment is difficult because a long section of the reference genome is missing in the sample. Therefore, processor 101 uses a different approach without alignment.

In some examples, the strands 112 on the flow cell 111 are strands of RNA, so that the sequencing data represents expression data indicative of how a DNA sequence is expressed into RNA. From the expression data, processor 101 can then detect deletions in the DNA sequence when compared to a reference sequence by identifying which regions of the reference genome are not expressed.

### Method

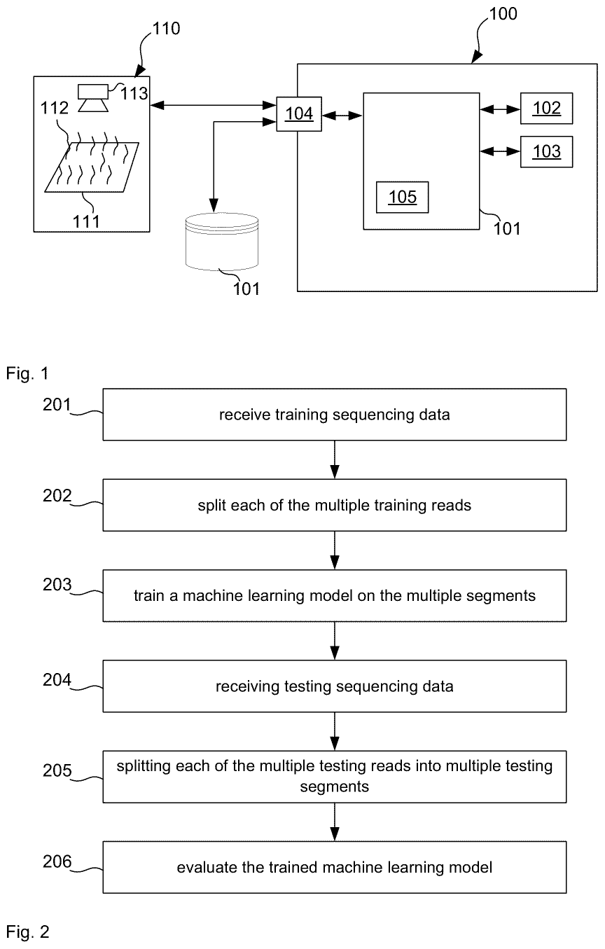

FIG. 2 illustrates a method 200 as performed by processor 101 for detecting deletion in a DNA sequence. Method 200 comprises receiving 201 training sequencing data. The training sequencing data comprises multiple training reads from sequencer 110. The training reads are separated into two sets and labelled. The first set is associated with gene sequences with deletion (labelled ‘1’ for example) and the second set is associated with gene sequences without deletion (labelled ‘0’ for example). The label may also indicate whether an individual subject has a disease or is healthy.

Processor 101 splits each of the multiple training reads into multiple training segments shorter than the training reads. For example, the training reads may be 150 bp long while the segments are between 10 and 50 bp long.

Processor 101 then trains a machine learning model on the multiple segment. Once the training is complete and the trained machine learning model stored on data memory 103, processor 101 receives 204 testing sequencing data comprising multiple testing reads. In some examples, the testing sequencing data is from a sample from a patient who is to be diagnosed.

Processor 101 again splits 205 each of the multiple testing reads into multiple testing segments; and evaluate 206 the trained machine learning model to the multiple testing segments to detect deletion in the testing sequencing data.

### Machine Learning Model

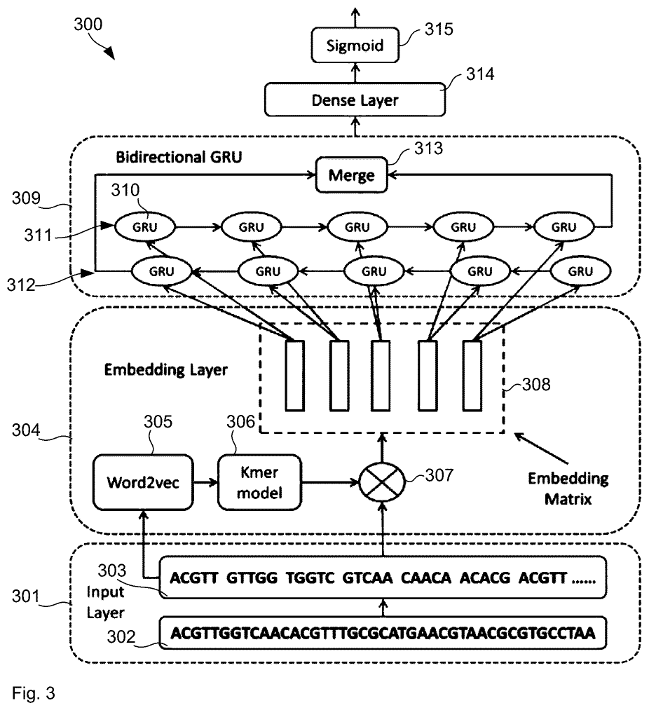

FIG. 3 illustrates a machine learning model 300 in the form of a neural network. In this example, the machine learning model 300 comprises an input layer 301 and embedding layer 304, a bidirectional gated recurrent unit (GRU) 309, dense layer 312 and sigmoid output 313.

Input layer 301 shows an example input read 302 and a set of segments 303 after the processor 101 has split the read 302. Embedding layer comprises a word2vec module 305 and a kmer model 306, both of which may be omitted in some examples. Word2vec is a technique for natural language processing. The word2vec algorithm uses a neural network model to learn word associations from a large corpus of text. Once trained, such a model can detect synonymous words or suggest additional words for a partial sentence. Here, Word2vec can be applied to segments of reads.

Further, embedding layer 304 comprises an embedding matrix 308. An embedding matrix is a linear mapping from the original space (one-of-k) to a real-valued space where entities can have meaningful relationships. Just like other matrices in neural network, the embedding matrix can be trained as well. So here, the original space may be the space of all possible kmers and the embedding matrix maps that space to a real valued space.

The real valued results from embedding layer are used in the Bidirectional GRU. This involves multiple individual GRUs 310 that each receive the output of the embedding layer 304. In this example, there are two strings of GRUs 311 and 312 and each string comprises multiple GRUs connected in series, such that the output of one GRU in the string serves as an input to a ‘downstream’ GRU. The results from both strings 311 and 312 are merged by a merge operation 313. The result of the merge operation 313 is then provided to a dense layer 314 comprising multiple neurons (not shown). In a dense layer, each neuron in the layer receives an input from all the neurons present in the previous layer—thus, they're densely connected. In other words, the dense layer is a fully connected layer, meaning all the neurons in a layer are connected to those in the next layer. More details on the model can be found in Zhen Shen, Wenzheng Bao & De-Shuang Huang, “Recurrent Neural Network for Predicting Transcription Factor Binding Sites” in Nature Scientific Reports (2018) 8:15270, which is included herein by reference.

Finally, a sigmoid function 315 calculates an output classification/label based on the result of the dense layer. This output may be a disease indicator or the presence of a deletion.

Direct Learning

While FIG. 3 shows an embedding layer, it is also possible to learn on the segments without the embedding layer 304, such as using the one-hot encoding {‘A’:0,‘C’:1,‘G’:2,‘T’:3,‘N’:4}.

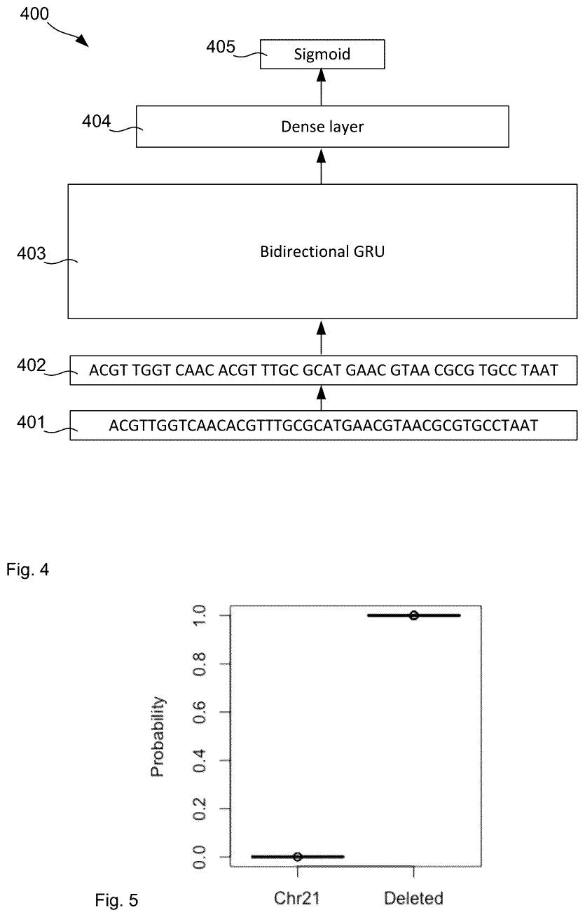

FIG. 4 illustrates a resulting machine learning model 400, where the input read 401 is split into segments 402 and the encoded segments 402 are used directly in the bidirectional GRU 403. This is particularly useful in this case of detecting deletions because the presence or absence of segments is closer to a binary decision than in up- or down-regulation.

### Example

This disclosure sets out how differential analysis can be done by machine learning neural networks at the DNA genomics level. For example, consider chromosome 21 in a healthy subject's genome. At a point in time, two DNA pieces on the chromosome are deleted. The deleted DNA could lead to diseases.

The methods disclosed herein use machine learning to “remember” those deleted regions. The example below is heavily simplified to provide an explanation of the process:

Sequence of chromosome 21: 0123456789 Each number represents the position of a specific nucleotide. The numbers are used for the nucleotides going forward for illustrative purposes.

In this example, k-mer length is set to 4. This will result in the following k-mers from the healthy genome and a binary label. Binary label 0 means “healthy”:

| Segment | Label |
|---|---|
| 0123 | 0 |
| 1234 | 0 |
| 2345 | 0 |
| 3456 | 0 |
| 4567 | 0 |
| 5678 | 0 |
| 6789 | 0 |

Now there is a deletion of “23456”, which results in the following k-mers from this deleted region. Binary label 1 means “disease”.

| Segment | Label |
|---|---|
| 2345 | 1 |
| 3456 | 1 |

Once the neural network is trained, processor 101 can use “789” as a testing segment. The result is a very low probability (about 0.01), indicating this region does not overlap with diseases For testing segment “2345”, the network provides me a very high probability (about 0.99), indicating this region is overlapped with diseases.

In this sense, the network acts like a “dictionary”, memorising what is healthy (0) and what is disease (1) using a bidirectional GRU. The GRU is bidirectional because the k-mers can be oriented from left-to-right and right-to-left.

### Implementation

In one example, the disclosed method is implemented based on Kaggle using Keras, such as by:
model=Sequential( )
model.add(Embedding(max_fatures, embed_dim, input_length=X.shape[1]))
model.add(SpatialDropout1D(0.4))
model.add(LSTM(lstm_out, dropout=0.2, recurrent_dropout=0.2))
model.add(Dense(2,activation=‘softmax’))
model.compile(loss=‘categorical_crossentropy’,
optimizer=‘adam’,metrics=[‘accuracy’])

In another example, the model uses a one-dimensional convolutional layer. The Keras solution look like:
model.add(conv1D(4,L,input_shape=x.shape[1:],activation=‘relu’))
model.add(Bidirectional(GRU(512, return_sequences=True)))
model.add(Bidirectional(GRU(512)))
model.add(Dense(512,activation=‘relu’))
model.add(Dense(1,activation=‘sigmoid’))

The proposed model was able to achieve 99% training accuracy, after 4 epochs using the standard gradient descent. There was no attempt at preventing overfitting such as inserting dropout layers. The output of the model is a sigmoid (could also be softmax), generating a probability for each DNA sequence.

FIG. 5 illustrates a sigmoid curve with a threshold 0.50 which may achieve a sufficient ROC.

As previously mentioned, processor 101 also comprises GPU 105, which may also be located externally to processor 101. In one example, the training or evaluation or both of the machine learning model is at least partly performed by the GPU 105. The advantage is that GPUs are designed with a high degree of parallelism, which means the training of the neural network can be completed within a significantly reduced time frame.

### Experiment

The disclosed method was tested on:
Longer chromosomes (chr1 and chr18)
Various sequencing coverage (10×, 30×, 50× and 100×)
Numbers of regions (1 to 3)

The loss function, like before, is binary_crossentropy (https://www.il/losses/). Two hidden layers. The implementation may convert the sequencing data to one-hot encoding using the rule: {‘A’:0,‘C’:1,‘G’:2,‘T’:3,‘N’:4}

The accuracy was good and the separation from chr18 was good, much like chr21. In order to improve robustness of the model, memory usage can be reduced. For example, instead of reads from the entire genome, it may be possible to load a random subset of the genome. Further, the model can be expanded and more hidden layers may improve the result.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A computer-implemented method for detecting deletion in a gene sequence, the method comprising:
receiving training sequencing data, the training sequencing data comprising multiple unaligned training reads associated with gene sequences with deletion and gene sequences without deletion;
splitting each of the unaligned multiple training reads into multiple training segments shorter than the training reads;
training a machine learning model on the multiple segments;
receiving testing sequencing data comprising multiple unaligned testing reads;
splitting each of the multiple unaligned testing reads into multiple testing segments; and
evaluating the trained machine learning model to the multiple testing segments to detect deletion in the testing sequencing data, wherein
the training sequencing data and the testing sequencing data comprise RNA reads and the deletion is in a genome of a subject,
the machine learning model is a neural network comprising a bidirectional gated recurrent unit to process forward and reverse read directions of the training sequencing data and the testing sequencing data, and
the method further comprises encoding the multiple segments and using the encoded segments directly as an input to the bidirectional gated recurrent unit.

2. The method of claim 1, wherein the training segments and the testing segments are k-mers.

3. The method of claim 1 or 2, wherein the testing sequencing data is generated by a sequencer.

4. The method of claim 3, wherein the testing sequencing data is provided in a FASTQ file from the sequencer.

5-8. (canceled)

9. The method of any one of the preceding claims, wherein the method further comprises performing one or more steps of the method on a graphics processing unit.

10. The method of any one of the preceding claims, wherein the method further comprises detecting a disease based on the deletion.

11. The method of claim 10, wherein detecting the disease is an output of the trained machine learning model.

12. The method of any one of the preceding claims, wherein the training sequencing data and the testing sequencing data is obtained by sequencing by synthesis.

13. (canceled)

14. The method of any one of the preceding claims, wherein the reads are between 100 and 200 base pairs long and the segments are between 4 and 100 base pairs long.

15. The method of claim 14, wherein the segments are between 4 and base pairs long.

16. Software that, when executed by a computer, causes the computer to perform the method of any one of the preceding claims.

17. A computer system for detecting deletion in a gene sequence, the computer system comprising:
data memory configured to store training sequencing data, the training sequencing data comprising multiple training reads associated with gene sequences with deletion and gene sequences without deletion;
a processor configured to:
split each of the multiple training reads into multiple training segments shorter than the training reads;
train a machine learning model on the multiple segments;
receive testing sequencing data comprising multiple testing reads;
split each of the multiple testing reads into multiple testing segments; and
evaluate the trained machine learning model to the multiple testing segments to detect deletion in the testing sequencing data, wherein
the training sequencing data and the testing sequencing data comprise RNA reads and the deletion is in a genome of a subject
the machine learning model is a neural network comprising a bidirectional gated recurrent unit to process forward and reverse read directions of the training sequencing data and the testing sequencing data, and
the method further comprises encoding the multiple segments and using the encoded segments directly as an input to the bidirectional gated recurrent unit.
